(19) United States
(12) Patent Application Publication
(10) Pub. No.: US 2016/0249833 A1
(43) Pub. Date: Sep. 1, 2016

(54) METHOD AND APPARATUS FOR MONITORING QUALITY OF A DYNAMIC ACTIVITY OF A BODY

(71) Applicant: DORSAVI PTY LTD, Melbourne East, Victoria (AU)

(72) Inventors: Daniel Matthew Ronchi, Victoria (AU); Andrew James Ronchi, Victoria (AU); Edgar Charry, Victoria (AU); Wenzheng Hu, Victoria (AU); Michael Panaccio, Victoria (AU); Muhammad Umer, Victoria (AU)

(21) Appl. No.: 15/023,179

(22) PCT Filed: Sep. 19, 2014
(86) PCT No.: PCT/AU2014/000926

§ 371 Date: Mar. 18, 2016

(30) Foreign Application Priority Data
Sep. 19, 2013 (AU) 2013903605

(51) Int. Cl. A61B 5/11; G01P 15/14; G01C 22/00; G01P 15/02

(52) U.S. Cl. CPC A61B 5/1118; A61B 5/1123; G01P 15/02; G01P 15/14; G01C 22/006

(57) ABSTRACT

Apparatus is disclosed for monitoring, measuring and/or estimating metrics and/or combinations of the metrics associated with Quality of a dynamic activity of a body or body part of a vertebral mammal. The apparatus includes at least one inertial sensor for measuring relative to a first frame of reference acceleration and/or rotation data indicative of the Quality of a dynamic activity and for providing the acceleration and/or rotation data. The apparatus also includes a memory device adapted for storing the acceleration and/or rotation data, and a processor adapted for processing the acceleration and/or rotation data to evaluate one or more biomechanical metrics associated with Quality of the dynamic activity that correlates to the data. The processor may be configured to execute at least one algorithm for evaluating the one or more biomechanical metrics associated with quality of the dynamic activity. A method for monitoring, measuring and/or estimating metrics and/or combinations of the metrics associated with Quality of a dynamic activity of a body or body part of a vertebral mammal is also disclosed.

### CROSS-REFERENCE TO RELATED APPLICATIONS

The present invention is related to the following patent applications assigned to the present applicant, the disclosures of which are incorporated herein by cross reference.

PCT/AU2013/000814 filed on 24 Jul. 2013 and entitled Method and apparatus for measuring reaction forces.

PCT/AU2013/001295 filed on 8 Nov. 2013 and entitled Method and apparatus for monitoring deviation of a limb.

PCT/AU2014/000426 filed on 14 Apr. 2014 and entitled Method and Apparatus for Monitoring Dynamic Status of a Body.

### TECHNICAL FIELD

The present invention relates to a method and apparatus for monitoring, diagnosing, measuring and/or providing feedback on metrics associated with Quality of a dynamic activity of a body or body part of a vertebral mammal.

### BACKGROUND OF INVENTION

The present invention will hereinafter be particularly described with reference to measurement of biomechanical metrics relating to Quality of a dynamic activity such as walking and/or running. Nevertheless it is to be appreciated that the present invention is not thereby limited to measurement of such dynamic activity.

Runners at different skill levels, from recreational to professional, have a need for immediate and easy access to information about their running style. Objective information relating to biomechanical parameters such as ground contact time, knee deviation, stride length etc. may be used for both performance improvement and injury prevention.

Existing systems that report on similar biomechanical measurements are either laboratory-based or require direct observation of a subject by video, infrared signals or other means that are not fully ambulatory. The apparatus of the present invention may be configured to provide a system for measurement of running quality that may be completely ambulatory, personalized and easy to use. The system may be used by individuals, recreation and professional runners alike.

The method and apparatus of the present invention may monitor and/or estimate multiple biomechanical metrics and/or parameters and/or various combinations of the metrics associated with the dynamic activity of the body or body part. Examples of biomechanical metrics associated with Quality of a dynamic activity such as walking and/or running that may be monitored include a measure of airborne time, speed, vertical, medio-lateral and anterior-posterior speeds, displacement, distance, stride length, stride rate, knee height, knee deviation, ground contact time, foot strike type, minimum toe clearance, acceleration and/or angular rate of change of a body or body part, vertical, horizontal, rotational 3D forces, timing of forces and impact and vibration applied to and/or experienced by the body or body part.

A reference herein to a patent document or other matter which is given as prior art is not to be taken as an admission that that document or matter was known or that the information it contains was part of the common general knowledge in Australia or elsewhere as at the priority date of any of the disclosure or claims herein. Such discussion of prior art in this specification is included to explain the context of the present invention in terms of the inventor's knowledge and experience.

Throughout the description and claims of this specification the words “comprise” or “include” and variations of those words, such as “comprises”, “includes” and “comprising” or “including, are not intended to exclude other additives, components, integers or steps.

### SUMMARY OF INVENTION

According to one aspect of the present invention there is provided apparatus for monitoring, measuring and/or estimating metrics and/or combinations of the metrics associated with Quality of a dynamic activity of a body or body part of a vertebral mammal, said apparatus including:
at least one inertial sensor for measuring relative to a first frame of reference acceleration and/or rotation data indicative of said Quality of a dynamic activity and for providing said acceleration and/or rotation data;
a memory device adapted for storing said acceleration and/or rotation data; and
a processor adapted for processing said acceleration and/or rotation data to evaluate one or more biomechanical metrics associated with Quality of said dynamic activity that correlates to said data.

The apparatus may optionally include a magnetic field sensor for measuring a magnetic field around the body or body part and for providing data indicative of the magnetic field. The dynamic activity to be monitored may include walking and/or running.

The processor may be configured to execute at least one algorithm for evaluating the one or more biomechanical metrics associated with quality of the dynamic activity. The at least one algorithm may be adapted to evaluate the or each biomechanical metric based on features of a signal detected by a Wavelet transform of the data.

The Wavelet Transform may be adapted to detect local features in a time-domain of a signal measured by the at least one inertial sensor. The local features may include specific peaks, troughs and/or slope of the signal being features related to known events, such as heel strike, toe off and/or knee deviation.

The Wavelet Transform may be adapted to decompose the signal into approximation decompositions and detail decompositions associated with the local features, being shifted and/or scaled versions of a mother wavelet.

In order to provide robust and real-time detection of local features, the present invention may comprise a wavelet-based algorithm. The algorithm may rely on typical frequency bands specific to a signal for the activity being monitored.

The biomechanical metrics associated with quality of the dynamic activity may include a measure of airborne time, speed, vertical, medio-lateral and anterior-posterior speeds, displacement, distance, stride length, stride rate, knee height, knee deviation, ground contact time, foot strike type, minimum toe clearance, acceleration and/or angular rate of change of the body or body part, vertical, horizontal, rotational 3D forces, timing of forces and impact and vibration applied to and/or experienced by the body or body part. The biomechanical metrics may be used to provide a scoring system for quality of the dynamic activity. Preferably two or more biomechanical metrics may be used in combination to provide a score or measure of quality of a dynamic activity of a body or body part of a vertebral mammal.

The or each metric or a related scoring system associated with quality of the dynamic activity may be assessed with reference to a preferred range or threshold of values. One measure of Quality of a running event may include the status of bio-mechanical metrics relative to known, implied or ideal ranges or thresholds. A variation in the metrics beyond these ranges or thresholds may indicate potential biomechanical issues that may relate to injury or other problems or may indicate degradation of overall performance when running.

In the context of the present embodiment, a preferred range of ground contact times for optimal running may be 180-200 milliseconds. Stride rate may be optimal at substantially 170-190 steps per minute, preferably 180 steps per minute. Stride length may be optimal when the ratio of stride length to leg length lies substantially in the range 2.6 and 2.9. GRFs may be optimal when an Absolute Symmetry Index (ASI), which computes level of asymmetry between forces on the left (GRF L) and right (GRF R) legs, lies substantially between ±10%. ASI is defined as 100*(GRF L−GRF R)/(GRF L+GRF R)/2. In addition, an accumulation of each footfall's GRF over a sprint or jog may provide a meaningful scoring measure for runners during a single run and for tracking different runs over time. For example, a measure of ‘load total’ for a jogging session may be calculated by taking the GRF for each stride and summing them all for the jog period.

The at least one inertial sensor may include an accelerometer. The accelerometer may be adapted for measuring acceleration along one or more orthogonal axes. The at least one inertial sensor may include a gyroscope and/or a magnetometer. The present invention may evaluate metrics associated with the body part by using two inertial sensors such as accelerometers. The present invention may avoid a need to transform sensor measurements to a global frame of reference by using an additional sensor such as gyroscope and/or magnetometer.

The body of the mammal may include lower limbs such as tibias and the at least one inertial sensor may include a wireless acceleration sensor adapted to be placed on each tibia.

The at least one inertial sensor may include an analog to digital (A to D) converter for converting analog data to a digital domain. The A to D converter may be configured to convert an analog output from the wireless acceleration sensor to digital data prior to storing the data. The apparatus may include means for providing feedback to a subject being monitored.

An additional sensor, such as gyroscope or magnetometer may be used to provide angular displacement of the body part for an event associated with a running activity, such as knee deviation when the leg hits the ground or knee range of movement.

The algorithm may be adapted to integrate rotation and/or magnetic field data over a period of time to provide angular displacement. The algorithm may be adapted to integrate the data over a period of time to provide the angular displacement (e).

The events to be monitored may manifest while performing physical activities and/or movements including activities and/or movements such as walking, running and/or sprinting, hopping, landing, squatting and/or jumping. Some activities may include movements of limbs of interest including legs. Other activities such as playing a game of tennis may include movement of limbs of interest including arms.

According to a further aspect of the present invention there is provided a method for monitoring, measuring and/or estimating metrics and/or combinations of the metrics associated with Quality of a dynamic activity of a body or body part of a vertebral mammal, said method including:
using at least one inertial sensor to measure relative to a first frame of reference acceleration and/or rotation data indicative of said Quality of a dynamic activity and to provide said acceleration and/or rotation data;
storing said acceleration and/or rotation data in a memory device; and
processing said acceleration and/or rotation data by a processor to evaluate one or more biomechanical metrics associated with Quality of said dynamic activity that correlates to said data.

### BRIEF DESCRIPTION OF DRAWINGS

FIGS. 1(a) to 1(g) show examples of running events and associated accelerometer data from a tibia;

FIG. 2 shows placement of sensors on a medial part of the tibia;

FIG. 3 shows one form of apparatus according to the present invention;

FIG. 4a shows a transversal plane cut of the tibia highlighting transformation of sensor data from sensor frame B to frame C;

FIG. 4b shows transformation of sensor data from frame C to global frame O;

FIG. 5 shows a flow chart of a data processing algorithm for obtaining a measure of quality of running;

FIG. 6 shows a flow chart of a Wavelet-based algorithm being used to detect features of running events;

FIG. 7 shows an acceleration signal and four daughter wavelets;

FIGS. 8(a) to 8(d) show examples of sprinting data from four different subjects and detected gait events;

FIG. 9 shows synchronized accelerometer and force plate data portraying delay δ for a “toe off” event measured by a sensor;

FIG. 10 shows a scatter plot of delay δ versus speeds for data obtained from six subjects and a linear best-fit;

FIG. 11 shows an example of ground contact time measured over time from a running subject;

FIG. 12 shows an example of angular measurements of knee deviation in sagittal and medio-lateral planes and associated tibial acceleration data;

FIG. 13 shows a scatter plot of knee height versus peak acceleration for data obtained from three subjects;

FIGS. 14(a) and 14(b) show average height for the left and right knees for a subject and knee height asymmetry index for the same subject;

FIGS. 15(a) and 15(b) show scatter plots of maximum acceleration slope and maximum binned acceleration slope for three subjects;

FIG. 16 shows plots of speed measured via sensors and GPS;

FIG. 17 shows stride length for one subject during a run; and

FIG. 18 shows a scatter plot of acceleration versus speed during Flat foot events.

### DETAILED DESCRIPTION

A preferred embodiment of the present invention includes one or more wireless inertial sensors adapted to be placed on one or both lower limbs such as on each tibia. In some embodiments the one or more sensors may be associated or incorporated with the lower limbs by being attached to an ankle or incorporated with footwear such as the sole of a shoe. The sensors may continuously measure inertial forces acting on the lower limbs during a running gait cycle. Metrics associated with running quality such as ground contact time and/or knee deviation may be computed from models derived from past data and/or specific features from the sensor signals. The specific features may include peaks, troughs and/or the slope of acceleration signals measured by the inertial sensor placed on the lower limb such as on the tibia. The specific features may be physically related to known gait events, such as heel strike or toe off.

Running quality may be objectively measured by analysing detected gait events indicating in terms of their magnitude, relative difference between left and right feet, timing and/or duration. For example ground contact time may be defined as time between heel strike and toe off gait events while knee deviation may be defined as magnitude of knee angulation between foot strike and toe off time.

A preferred embodiment of the present invention will be described below with a focus on a running activity. A running activity may be divided into two basic phases: a stance phase and a swing phase. The stance phase occurs when the foot is in contact with the ground, while the swing phase occurs when the foot is in the air. Running is characterized by the fact that at some point in the running cycle, both feet are in the air simultaneously.

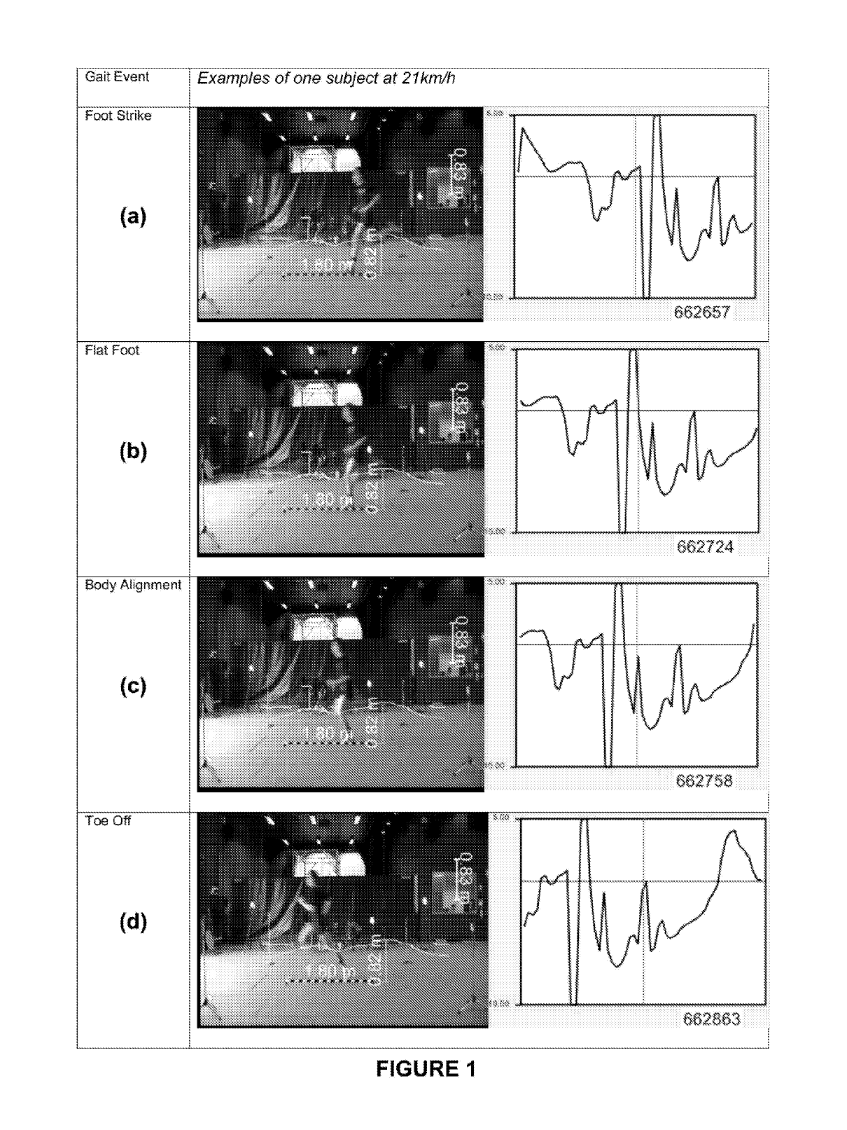

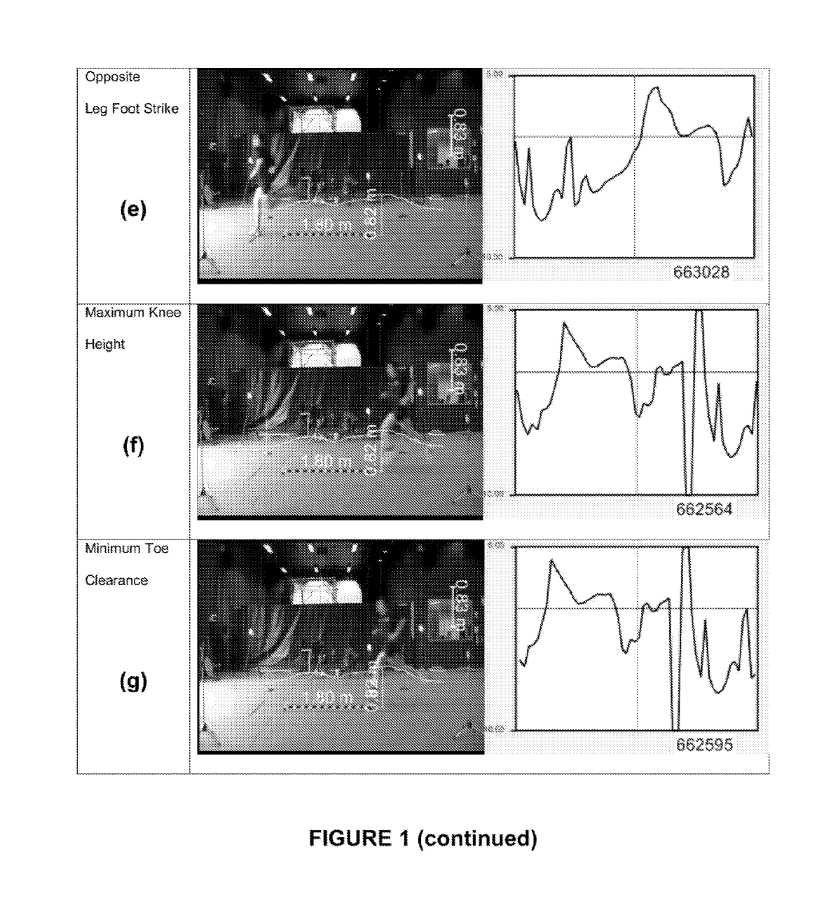

FIGS. 1(a) to 1(g) show video snapshots of gait events from one subject running at 21 km/h. The gait events shown in FIGS. 1(a) to 1(g) are Foot Strike (FS), Flat Foot (FF), Body Alignment (BA), Toe Off (TO), Opposite Foot strike (OFS), Maximum Knee Height (MKH) and Minimum Toe Clearance (MTC) respectively.

The acceleration signals monitored by an inertial sensor placed on the tibia of a subject during running may be modelled as a quasi-periodic stochastic process, with variable temporal events that relate to gait events as outlined above. Automatic and reliable detection of gait events may be critical to providing real-time information related to different characteristics of the subject's gait pattern during walking or running. For example, this information may be used to derive ground contact time, ground reaction forces, or knee height. Consequently, feedback may be provided to the subject, so that the subject may modify his or her technique or training according to goals and experience.

### Feature Detection

Running events may be uniquely identified in the time domain by a set of wavelets. A Wavelet Transform may detect local features of different frequencies in the time-domain. The wavelet transform may decompose a time domain signal into shifted and scaled versions of a “mother” wavelet or into approximation and/or detail decompositions.

### Running Quality

During running, contact time may provide a measure of running quality as it is directly related to magnitude of power generated in an anterior-posterior plane. With a relatively low contact time, a runner may be required to exert more power to propel his/her leg forward. Contact time may therefore be considered as inversely proportional to metabolic cost of a run.

Existing methods of detecting contact time are based on direct and often subjective observation of a runner or by more sophisticated optical means. Consequently such methods may be highly restrictive in terms of the setting and surrounding environment where a test may be performed. In contrast, the method of the present invention may remove such constraints due to its completely ambulatory and objective nature. The method of the present invention may not be affected by gait variability and/or running speeds making it robust for a broad group of runners. After placing inertial sensors on a tibia, a runner may be free to choose a setting to run whether it is a treadmill or outdoors. Using aspects of the present invention, data samples may also be gathered for many consecutive steps as opposed to current techniques that allow only a limited number of steps to be captured and analysed.

Inward (valgus) or outward (varus) angulation of a knee is a known predictor of lower limb injuries such as shin splints in runners and in and other sports. Hence, in addition to contact time, presence and extent of valgus or varus tendency in a runner may be a useful metric of running quality. In order to provide information in real-time, automatic reporting of valgus or varus measures during a run may require additional information such as position of the knee at the instant of each foot strike.

### Apparatus

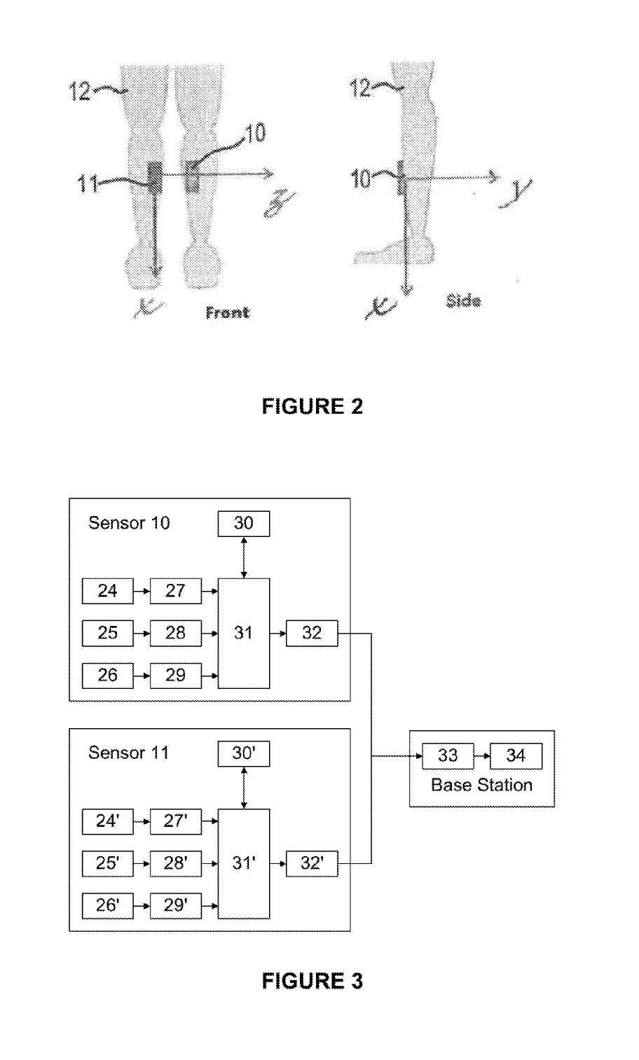

Apparatus according to the present invention may be placed on a body part such as a medial part of a tibia as shown in FIG. 2 to enable monitoring of 3D dynamics. The apparatus may include one or more inertial sensors such as accelerometers, gyroscopes and/or magnetometers as shown in FIG. 3. The apparatus may include a digital processing engine configured to execute one or more algorithms. The algorithm(s) may take account of variables such as movement of sensors during an activity relative to different frames of reference.

Referring to FIG. 2, one form of apparatus according to the present invention includes sensors 10, 11 placed along or in-line with tibial axes of the left and right legs of a human subject 12. Sensors 10, 11 are placed on the legs of subject 12 such that the frames of reference of sensors 10, 11 are defined by axes x,y,z with axes x,z being in the plane of FIG. 2 (front view) and axes x,y being in the plane of FIG. 2 (side view). For example measurement of Valgus or Varus may be defined as a rotation around the y axis.

Each sensor 10, 11 may include a rotation sensor such as a 1D, 2D or 3D gyroscope to measure angular velocity and optionally a 1D, 2D or 3D accelerometer to measure acceleration and/or a magnetic sensor such as a magnetometer to measure magnetic field. The positive axes on both legs may point up or down so that tibial acceleration may be measured in a vertical direction at least.

Referring to FIG. 3 each sensor 10,11 includes sensor elements 24, 25, 26 and 24′, 25′, 26′ for measuring acceleration, angular rotation and magnetic field data respectively. Data obtained from sensor elements 24,25,26 and 24′,25′,26′ is converted from an analog to digital format using Analog to Digital Converters (ADC) 27,28,29, and 27′, 28′, and 29′ respectively. The data may be held in digital memories 30 and 30′ for temporary analysis and/or storage. Coordination of data flow and processing of signals from sensor elements 24, 25, 26 and 24′, 25′, 26′ is performed by Central Processing Units (CPUs) 31 and 31′. Data measured via sensor elements 24, 25 and 26 and 24′, 25′ and 26′ may be sent via wireless transmitters 32, 32′ to a base station including remote receiver 33 and microprocessor 34. Microprocessor 34 is associated with remote receiver 33 and includes a digital processing engine for processing the data.

Digital memories 30, 30′ may include structure such as flash memory, memory card, memory stick or the like for storing digital data. The memory structure may be removable to facilitate downloading the data to a remote processing device such as a PC or other digital processing engine.

The digital memories 30, 30′ may receive data from sensor elements 24, 25, 26 and 24′, 25′, 26′. Each sensor element 24, 25, 26 and 24′, 25′, 26′ may include or be associated with a respective analog to digital (A to D) converter 27, 28, 29 and 27′, 28′, 29′. The or each A to D converter 27,28,29 and 27′,28′,29′ and memory 30, 30′ may be associated directly with sensor elements 24, 25, 26 and 24′, 25′, 26′ such as being located on the same PCB as sensor elements 24, 25, 26 and 24′, 25′, 26′ respectively. Alternatively sensor elements 24, 25, 26 and 24′, 25′, 26′ may output analog data to transmitters 32, 32′ and one or more A to D converters may be associated with remote receiver 33 and/or microprocessor 34. The one or more A to D converters may convert the analog data to a digital format or domain prior to storing the data in a digital memory such as a digital memory described above. In some embodiments microprocessor 34 may process data in real time to provide biofeedback to subject 12 being monitored.

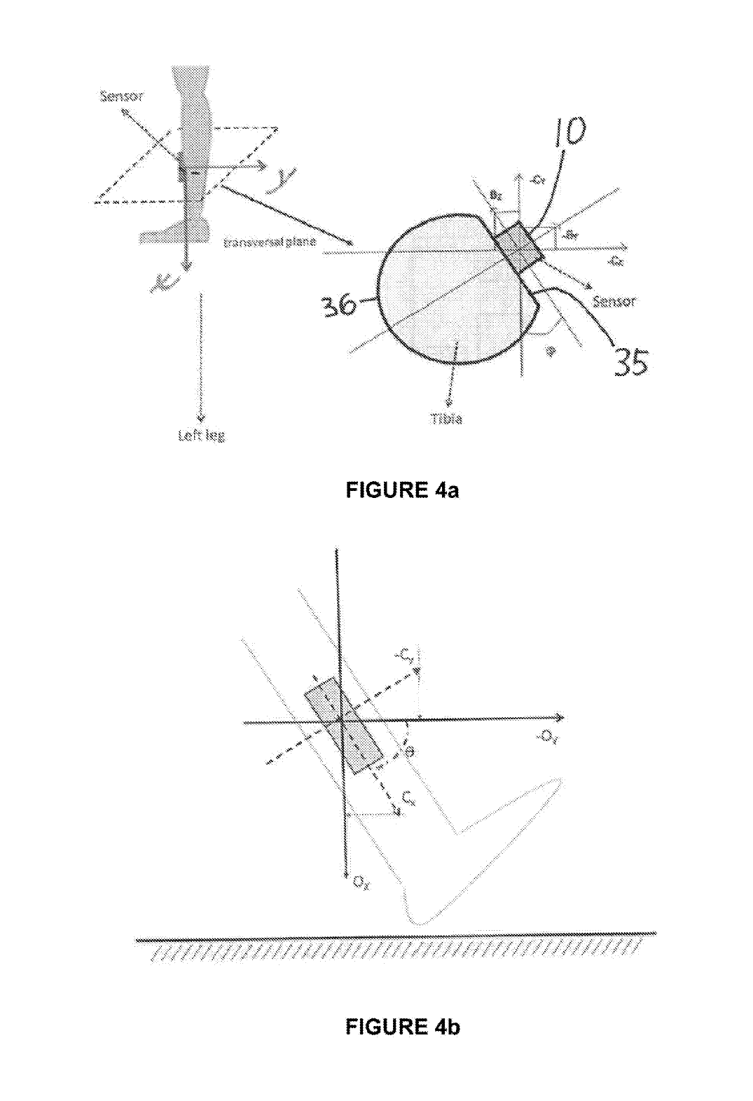

The digital processing engine associated with microprocessor 34 may include an algorithm for filtering and integrating gyroscope data, and transforming accelerations from a sensor element to a global frame perspective. The digital processing engine may perform calculations with the algorithm to adjust for limb bone angle such as 45° for the tibia of a human being following transformation of data from the frame of reference of each sensor 10 and 11 as shown in FIGS. 4aand 4b. Transformed gyroscope data may be filtered and integrated to obtain information on knee deviation status. The digital processing engine may also run algorithms to provide a score or measure over time based on one or a combination of the biomechanical metrics.

FIG. 4a shows a top-down cross-sectional view in the transversal plane of the left leg of subject 12 with sensor 10 placed on face 35 of tibia 36. The angle between face 35 on tibia 36 and the forward flexion plane is defined as φ. Angle φ may be approximately 45 degrees for an average subject but may vary a few degrees either side of the average value. Face 35 may provide a relatively stable platform for attachment of sensor 10. The frame of reference (B) for sensor 10 is therefore rotated relative to the frame of reference (C) of the mechanical axis of tibia 36 by the magnitude of angle φ. Flexion and lateral flexion are defined as rotations around axes Z and Y respectively.

Because measurements via sensor 10 are obtained in sensor reference frame B they must be converted to tibia reference frame C. The following equations may be used for this transformation:

Cy=By*cos(φ)+Bz*sin(φ) (1)

Cz=By*sin(φ)−Bz*cos(φ) (2)

wherein By Bz denote y and z components in sensor reference frame B, Cy and Cz denote y and z components in tibia reference frame C, and φ denotes the angle between sensor 10 on tibia 21 and the forward flexion plane.

Equations (1) and (2) above may be used to vector transform gyroscope signals {^{B}ωₓ, ^{B}ω_{Y} and ^{B}ω_{Z}} and optionally accelerometer signals {^{B}aₓ, ^{B}a_{Y} and ^{B}a_{Z}} obtained via sensor 10 in sensor reference frame B, to gyroscope signals {^{C}ωₓ, ^{C}ωw_{Y} and ^{C}ω_{Z}} and accelerometer signals {^{C}aₓ, ^{C}a_{Y} and ^{C}a_{Z}} respectively in mechanical or tibia reference frame C.

Following vector transformation, the gyroscope signals {^{C}ωwₓ, ^{C}ω^{Y} and ^{C}ω_{Z}} representing angular velocity may be integrated over a period of time t representing the duration of an activity such as squatting, hopping and/or running using the following equation to provide an integrated angular displacement (θ):

θ=∫₀^{t}ω·dt (3)

As a runner flexes the knee, movement such as medio/lateral deviation is measured with respect to mechanical or tibia reference frame (C). However, this value is transformed with respect to the visual reference frame of the tester, also known as the frontal or viewer plane to provide more intuitive results.

It is possible for the leg to rotate around the x-axis when the runner hops and lands. Hence, the visual impression of the lateral flexion will change if the rotation around the x-axis is not compensated. This effect is represented in equation 7, as it is used in the projection of the lateral flexion plane (θ_{z}) with respect to the frontal plane.

FIG. 4a also shows a projection of lateral flexion angle (θ_{Z}) onto the frontal or viewer plane together with a twist update. To project lateral flexion angle (θ_{Z}) onto the frontal or viewer plane the leg may considered to be a rigid rod with fixed joint on the ankle. The length of the rod may be normalized as 1. Angular displacement on the θ_{X} plane (caused by θ_{Y} and θ_{Z} only) may be determined by:

θₓ₀=atan(sin(θ_{Z})/tan(θ_{Y})) (4)

Actual twist movement θₓ₀ may be added to angular displacement θ_{X} to determine resultant angular displacement θ_{Xresultant}:

θₓᵣₑₛᵤₗₜₐₙₜ=θₓ+θₓ₀ (5)

One goal is to determine the terms A, B and C in order to calculate θ_{zAdjusted}. For this, the projection of θ_{Z} on θ_{X}, will result in A:

A=sin(θZ)/sin(θx0)*sin(θx) (6)

The projection of θ_{X} on θ_{Y} will determine B:

B=sin(θ_{Z})/sin(θₓ₀)*cos(θₓ) (7)

C is calculated assuming the length of the rod is 1:

C=sqrt(1−B²) (8)

Finally, calculate asin of A and C to obtain the drift adjusted θ_{Z} and projected onto the frontal plane as θ_{ZAdjusted}:

θ_{ZAdjusted}=a sin(A/C) (9)

The digital processing engine associated with microprocessor 34 may include a wavelet based algorithm for evaluating running events based on data from sensors 10, 11 and for providing information on running quality. In some embodiments a wavelet based algorithm may be included with Central Processing Units (CPUs) 31 and 31′ that perform preliminary processing of signals from sensor elements 24, 25, 26 and 24′, 25′, 26′.

The algorithm may use wavelet transforms to extract features from sensor signals based on multi-resolution analysis. The extracted features may be calibrated or correlated against known standards used for measuring running quality such as force plates, optical tracking systems, etc. Quality of running may be assessed with reference to implied or idealised thresholds or ranges associated with biomechanical metrics such as contact time, airborne time, knee deviation, knee height, stride rate, stride length, speed, distance, foot strike type and minimum toe clearance, obtained from known standards.

### Algorithms

### Data Flow and Gait Event Detection

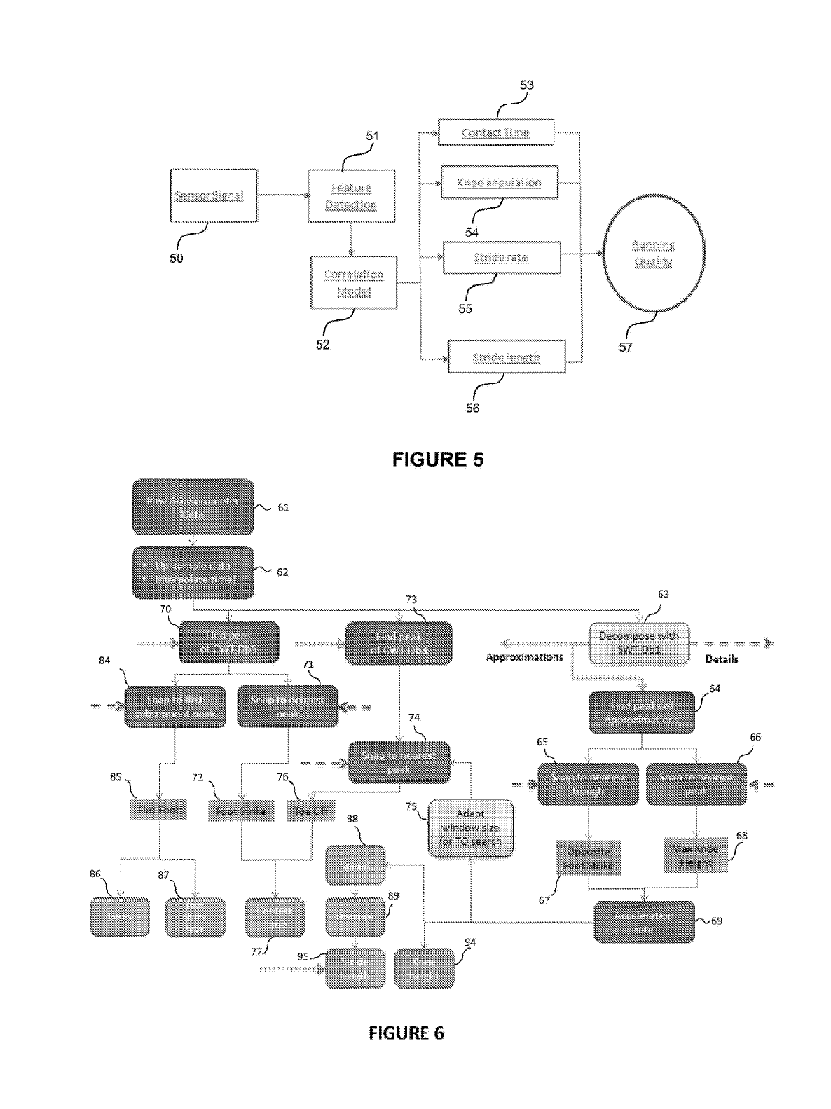

FIG. 5 shows an information processing flow diagram with an output 57 of correlations relevant to a measure of running quality. Sensor signal 50 is fed into feature detection algorithm 51. Feature detection algorithm 51 uses wavelet transforms to extract features in signal 50 based on multi-resolution analysis. The algorithm 51 may seek frequency bands that are inherently specific to running events. The frequency bands are due to variations in sensor signals based on a subjects gait variability and different speeds. A range of frequency bands and associated gait events that they are linked to is shown in Table 1 below.

TABLE 1

| | | | | | | Pseudo |
|---|---|---|---|---|---|---|
| Event | Type | Family | Order | Level | Scale | freq (Hz) |
| FS-IPA-FF | CWT | Daubechies | 5 | — | 21 | 23.7 |
| complex | | | | | | |
| OFS & | SWT | Daubechies | 1 | 7 | — | — |
| MKH | | | | | | |
| TO | CWT | Daubechies | 3 | — | 20 | 20.0 |

Features extracted from algorithm 51 in FIG. 5 may be correlated with metrics obtained empirically from a running event using known “Gold Standards” such as force plates and/or optical tracking systems. A model of these correlations 52 may be derived to estimate metrics relevant to quality of the running event such as contact time (53), knee angulation (54), stride rate (55) and stride length (56).

As discussed herein one measure of quality of a running event may include the status of each of the above metrics relative to known, implied or ideal ranges or thresholds. In the context of the present embodiment a preferred range of contact time 53 for optimal running is estimated to be substantially 180-200 ms. Stride rate 55 may be optimal at substantially 170-190 steps per minute, preferably 180 steps per minute. Stride length may be optimal when the ratio of stride length to leg length lies substantially in the range 2.6 and 2.9. GRFs may be optimal when an Absolute Symmetry Index (ASI), which computes level of asymmetry between Forces on the left (GRF L) and right (GRF R) legs, lies substantially between ±10%. ASI is defined as 100*(GRF L−GRF R)/(GRF L+GRF R)/2.

FIG. 6 depicts a flow diagram of an algorithm comprising blocks 61 to 77, 84-89 and 94-95. In Block 61 raw accelerometer data is collected from sensors 10, 11 placed on the tibias of subject 12.

Block 62 up-samples the data to 500 Hz to obtain greater resolution of sensor signals.

Block 63 decomposes a part of the sensor signals using a Stationary Wavelet Transform (SWT) of Daubechies family of order 1 and level 7. Block 63 generates approximation decompositions and detail decompositions using respective filter banks. The approximation decompositions may be used to find a low frequency region of the running cycle (refer daughter wavelet 79 in FIG. 7) which corresponds to a mid-swing phase and occurs near the Opposite Foot Strike (OFS) event. Detail decompositions on the other hand may detect peaks and troughs in the sensor signals (shown in FIG. 7 by “x” markers) and may be used to detect a region where it is likely that a foot strike occurs (corresponding to a high-frequency part of the signal).

Block 64 detects peaks of the approximation decomposition (refer FIG. 7—point marked with arrow 4), which represent the highest energy from that frequency band. Note that in FIG. 7, the daughter wavelet 79 of SWT−Db1 is a negative number.

Block 65 detects the nearest trough that corresponds to the Opposite Foot Strike (OFS) (refer Block 67).

Block 66 detects the nearest peak that corresponds to Maximum Knee Height (MKH) (refer Block 68).

Block 69 estimates the acceleration rate or slope between OFS and MKH.

Block 70 decomposes a part of the sensor signals using a Continuous Wavelet Transform (CWT) of Daubechies family of order 5 and scale 21 to detect the midpoint between FS and IPA (refer FIG. 7—point marked with arrow 1).

Block 71 detects the nearest peak between the midpoint of FS and IPA which corresponds to the points FS in FIG. 7 marked with a rectangle (refer Block 72).

Block 84 detects the nearest subsequent peak after the IPA, which corresponds to the point FF in FIG. 7 marked with a circle (refer Block 85).

Block 73 decomposes a part of the sensor signals using a Continuous Wavelet Transform (CWT) of Daubechies family of order 3 and scale 20 during the stance phase. The algorithm searches for the peak (refer FIG. 7—point marked with arrow 3) in this decomposition within a window calculated in Block 75 that will vary according to the slope of the acceleration signal.

Once the peak of the CWT in that window is found, Block 74 then detects the nearest peak that corresponds to a toe off (TO) event in the sensor signals (refer Block 76.

Running metrics may be estimated using acceleration values at gait event instants (blocks 67, 68, 85, 72 and 76) and their respective models (refer section on RUNNING METRICS). GRFs (86) and Foot Strike Type (87) may be found using Flat Foot event (85). Contact Time (77) may be estimated using Foot Strike (72) and Toe Off events (76). Knee Height (94) may be found with block 68. Speed (88) may be estimated using Acceleration Rate (69). Distance (89) and Stride Length (95) are derivatives of Speed.

FIG. 7 shows an example of an acceleration signal 78 and four daughter wavelets 79, 80, 81, 82 being used to detect running events. Wavelet 79 corresponds to Stationary Wavelet Transform (SWT) of Daubechies family of order 1 and level 7. Wavelet 79 may be used to find a low frequency region which corresponds to a mid-swing phase of the running cycle.

Wavelet 80 corresponds to a Continuous Wavelet Transform (CWT) of Daubechies family of order 5 and scale 21. Wavelet 80 may be used to detect the midpoint between FS and IPA (refer point marked with arrow 1).

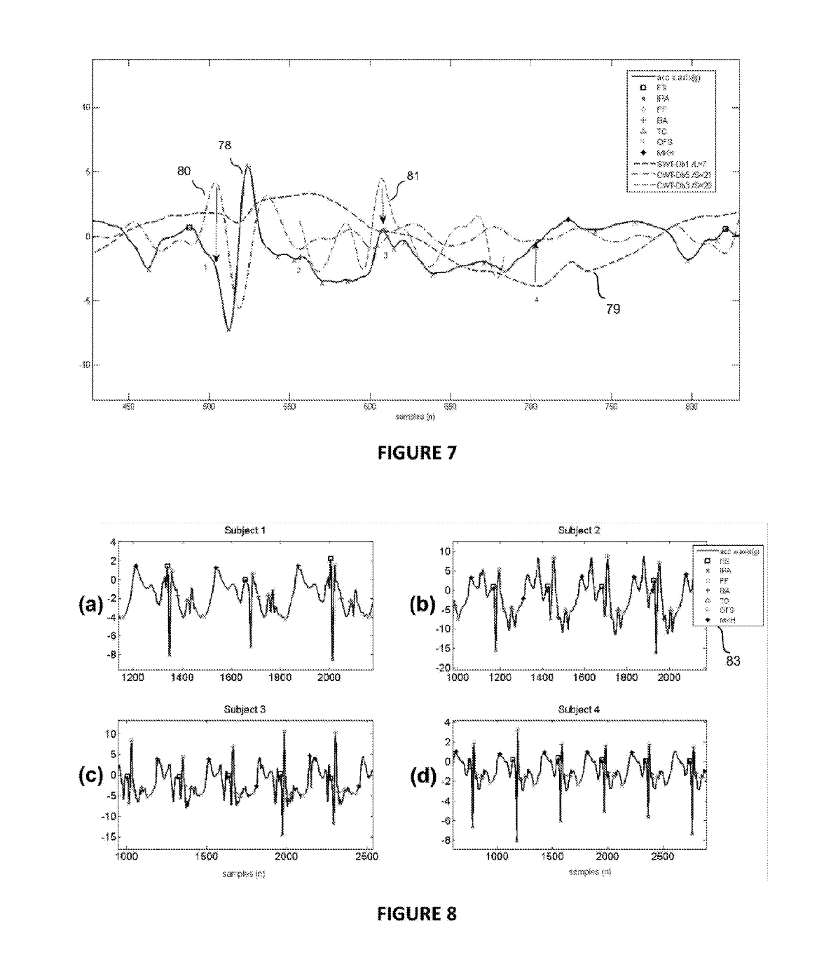

FIGS. 8(a) to 8(d) show sprinting data and detected events from subjects 1 to 4 respectively. The detected events FS, IPA, FF, BA, TO, OFS and MKH are marked with respective symbols as shown in legend 83. For example, FS is marked with a small rectangle. As may be observed, amplitude variations and non-stationary signals due to subject gait variability and variable speeds may be irrelevant for the algorithm, which may reliably detect the events notwithstanding the variations.

### Running Metrics

### Ground Contact Time

Ground contact time (t_{c})) measures the time spent during a stance phase. Specifically, contact time may be defined as the time elapsed between successive ipsilateral foot strike (FS) and toe off (TO) events during a gait cycle, i.e.:

t_{c}=t_{TO}−t_{FS} (10)

wherein t_{FS} and t_{TO} respectively represent instants of time when foot strike and toe off events occur.

The algorithm may compute t_{FS} and t_{TO} for each gait cycle of a run. However, contact time may not always be produced simply by taking a pairwise difference due to delays introduced by skin artefacts, time taken by sensors 10, 11 to process data and cushioning effects of shoes and terrain. In order to compensate for the latter delays, data from a force plate may be used to compare the contact time derived from sensors 10, 11.

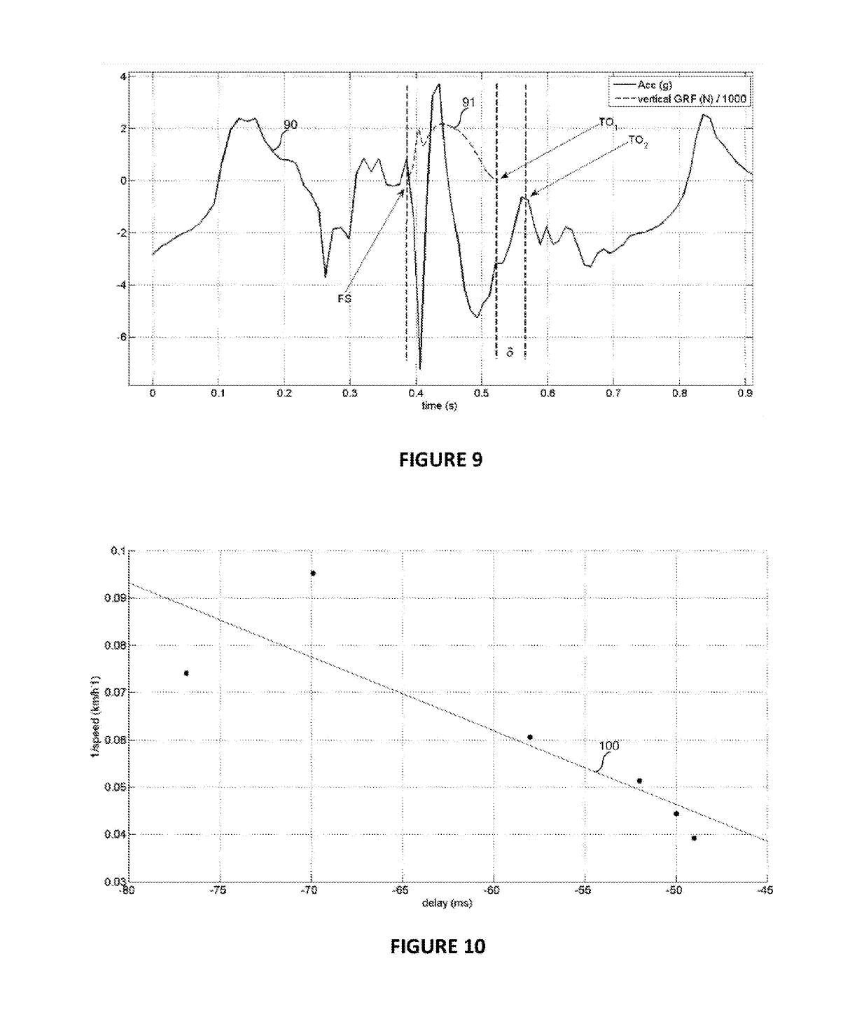

This is illustrated in FIG. 9 which shows traces of tibial acceleration 90 provided by sensors 10, 11 and vertical ground reaction force 91 provided by a force plate. FS is found on both traces according to Block 65 in FIG. 6, whereas TO is found visually on the accelerometer data (TO₂), being a local peak at the 0.57 s mark and on the force plate data (TO₁). The difference between TO₂ and TO₁ defines the overall delay δ.

FIG. 10 shows a scatter plot of delays versus the inverse of speeds from data for six subjects. The median values in this scatter plot are obtained to filter noisy results and a linear best fit 100 is shown. A correlation of −0.86 indicates that the faster is the speed, the lower is the delay. Hence a calculation of overall delay and compensated contact time t′_{c} may be given by the following equations:

δ=37.2+356.4/speed (11)

t′_{c}=t_{TO}−t_{FS}−δ (12)

wherein speed is measured in km/h and δ is measured in milliseconds.

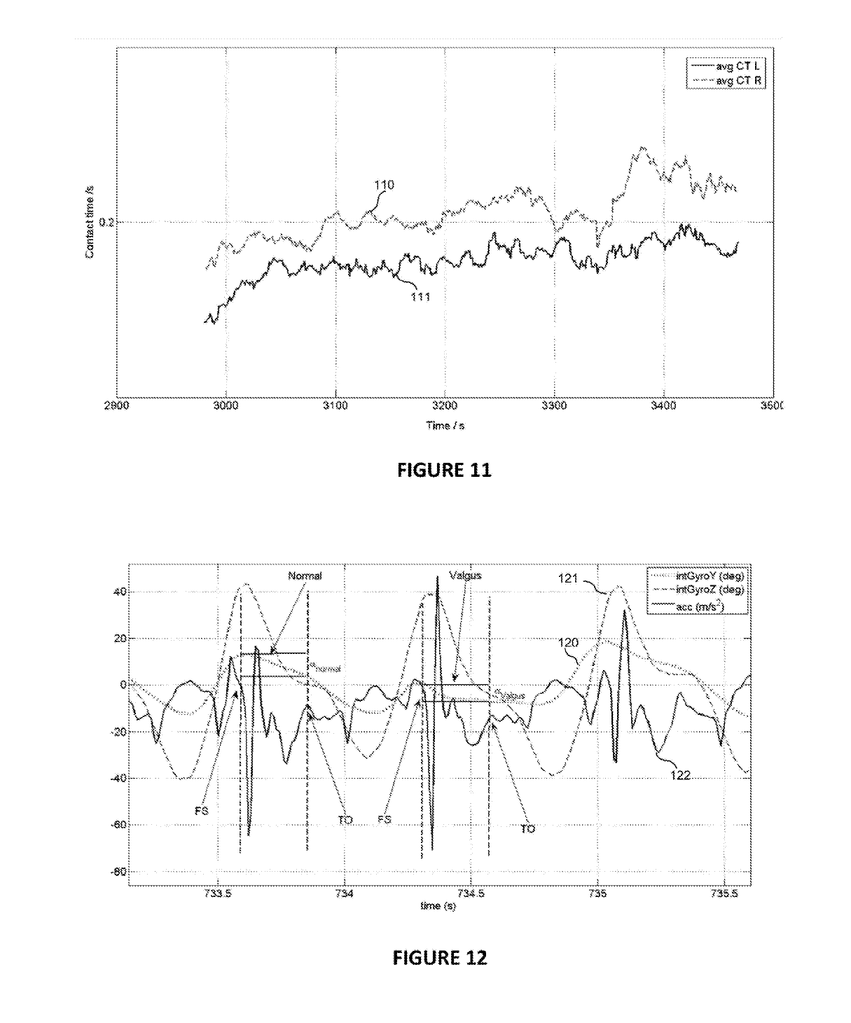

FIG. 11 shows traces 110, 111 of ground contact time (CT) for the right and left legs receptively of a subject over the course of a 1 kilometre run. It may be observed that the subject's right leg (trace 110) stays on the ground longer than the left leg (trace 111). As the subject runs, contact time increases from 180 ms to 220 ms.

### Knee Deviation

Automatic reporting of valgus or varus measures during a running event requires positional information of the knee at each foot strike instant. In the context of the present invention, an additional sensor, such as a gyroscope may be used to derive knee deviation and/or knee range of movement (ROM). Gyroscope data {gx, gy, gz} may be captured via sensors 10, 11, filtered to avoid data aliasing, buffered and transmitted wirelessly to the base station (33, 34).

Because sensors 10, 11 are placed on faces 35 of tibias 36, 45 degree angle (θ) compensation may be required to transform gyro signals from sensor frame B onto the medio-lateral and sagittal planes frame C for both left and right legs:

GyroY=gy·cos(θ)+gz·sin(θ) (13)

GyroZ=gy·sin(θ)+gz·cos(θ) (14)

The transformed gyroscope data GyroY and GyroZ is integrated over time. The initial angles g_{y0} and g_{z0 α} are set to zero, as measurements of knee deviation are taken with respect to gravity:

intGyroY=∫₀^{t}GyroY(t)·dt+g_{y0} (15)

intGyroZ=∫₀^{t}GyroZ(t)·dt+g _{z0} (16)

Due to cumulative errors arising from temperature variations and White Gaussian Noise (WGN), the integrated signals may drift randomly. Therefore, intGyroY and intGyroZ may be High-Pass-Filtered (HPF) to eliminate these errors. Since running and walking are cyclic applications high frequency components may be filtered out without compromising the integrity of knee deviation information. The employed filter may be an IIR (Infinite Impulse Response) Butterworth filter of order 4 and cut-off frequency of 0.1 Hz, as a lower order may be required to achieve a required pass band.

The model of the filter may be defined by:

$$y [ n ] = \frac{1}{a 0} ( b 0. x [ n ] + b 1. x [ n - 1 ] + … bP . x [ n - P ] - a 1. y [ n - 1 ] + a 2. y [ n - 2 ] + … aQ . y [ n - Q ] ] ) \qquad ( 17 )$$

wherein P=Q=4, x[n] and y[n] are input and outputs signals at time n respectively. In this application x[n] corresponds to intGyroY and intGyroZ at sample n, and y[n] is the filtered version of intGyroY andintGyroZ.

FIG. 12 depicts via trace 120 (intGyroY) an example of knee deviation in medio-lateral planes, wherein α_{Normal} and α_{Valgus} represent differences of the knee in the medio-lateral plane between foot-strike and toe-off. It may be observed that α_{Valgus} is a negative number, whereas α_{Normal} is positive when knee deviation is normal.

FIG. 12 also shows via trace 121 (intGyroZ) angular measurements in the sagittal plane, wherein the highest positive value corresponds to the FS instant in this example shown by one of the dashed vertical bars as well as tibial acceleration via trace 122.

### Knee Height

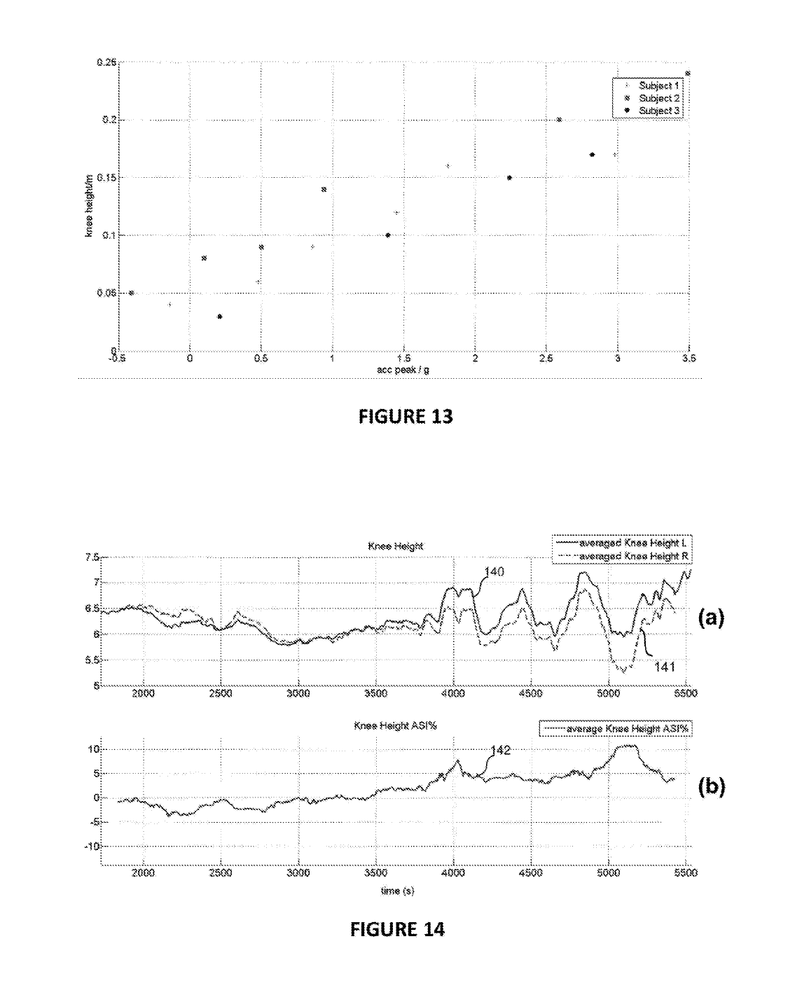

Automatic reporting of maximum knee height for both legs during a running event is measured through accelerometer data via sensors 10, 11. Peak acceleration may be correlated empirically with distance from the ground as depicted in FIG. 1(f). A linear model is depicted in the scatter plot of FIG. 13 with data from three subjects. Estimation may be performed by the following equation:

KneeHeight=0.047*peak_acc+0.056+CalKneeHeight (18)

wherein CalKneeHeight is knee height in meters of a subject when standing, peak_acc is acceleration in g's and KneeHeight is final height in meters. One example of knee height measurements is shown in FIG. 14(a), wherein a subject ran for 11 km. For the first half of the run (1500-3500 seconds), plots for left (140) and right (141) knees show good symmetry (average 0.5%), contrasting with asymmetry of 7% in average in the second half (refer plot 142 in FIG. 14(b)). This suggests that performance of the subject degraded quickly at the end of the run.

### Speed

Speed is measured as a maximum acceleration rate (MAR) between the opposite foot strike and maximum knee height. Physically, this may represent “kick” of the leg during the swing phase. The acceleration rate may be calculated as:

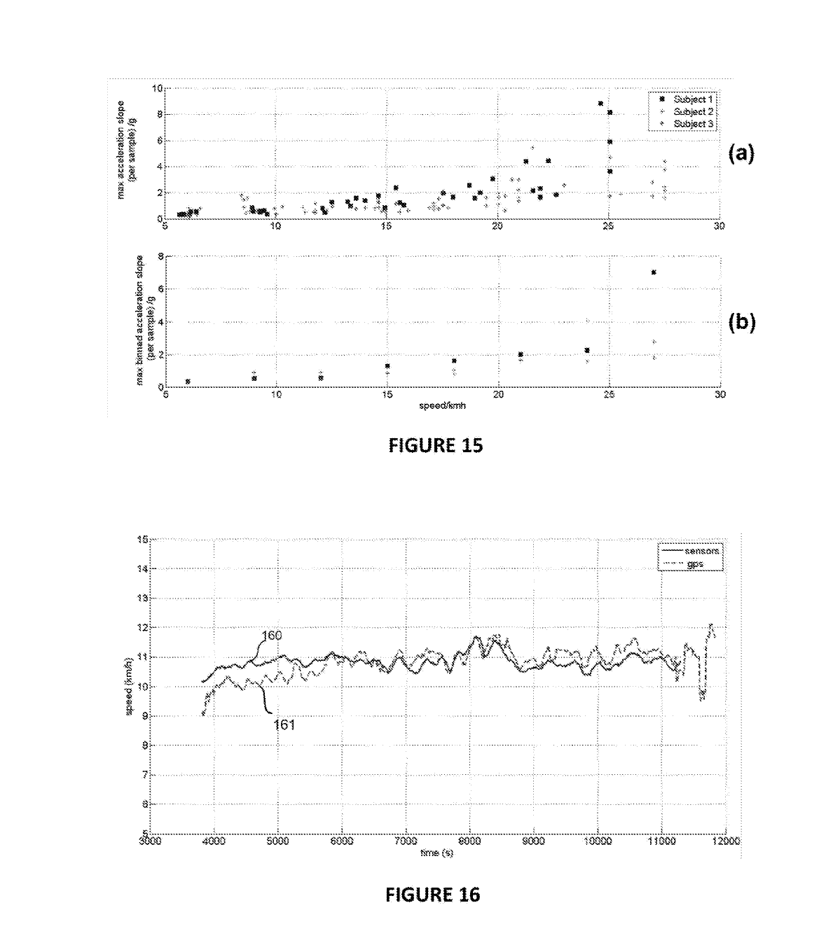

MAR=(acc_{MKH}−acc_{OFS})/(n_{MKH}−n_{OFS}) (19)

wherein acc_{MKH} and acc_{OFS} represent accelerations at MKH and OFS events and n_{MKH} and n_{OFS} represent samples at the same events. A scatter plot of the MAR from three subjects is shown in FIG. 15(a) and a version with median values (binned) of this scatter plot is shown in FIG. 15(b). The best fit model may be given by the equation:

Speed=9.35*MAR+4.69 (20)

FIG. 16 depicts a trace (160) of speed measured via sensors 10, 11 and a trace (161) of speed measured via GPS for one run of 24 km by one subject wearing a GPS unit on the wrist. Maximum speed error between both traces 160,161 is 0.5 km/h and there is good correlation between both systems.

### Stride Length

Stride length (SL) is calculated as:

D=∫₀^{t}Speed(t)·dt (21)

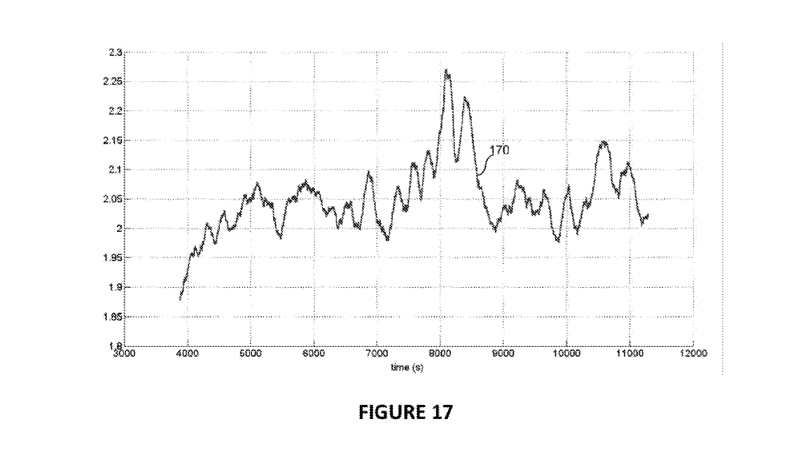

SL=D/N, wherein D is total distance in meters, N is total number of strides in a session and SL is stride length in meters. FIG. 17 shows a plot (170) of SL for one subject from a 24 km run wherein it may be observed that the subject is under-striding (SL<2.8*LL), wherein LL=0.95 m is the leg length.

### Foot Strike Type

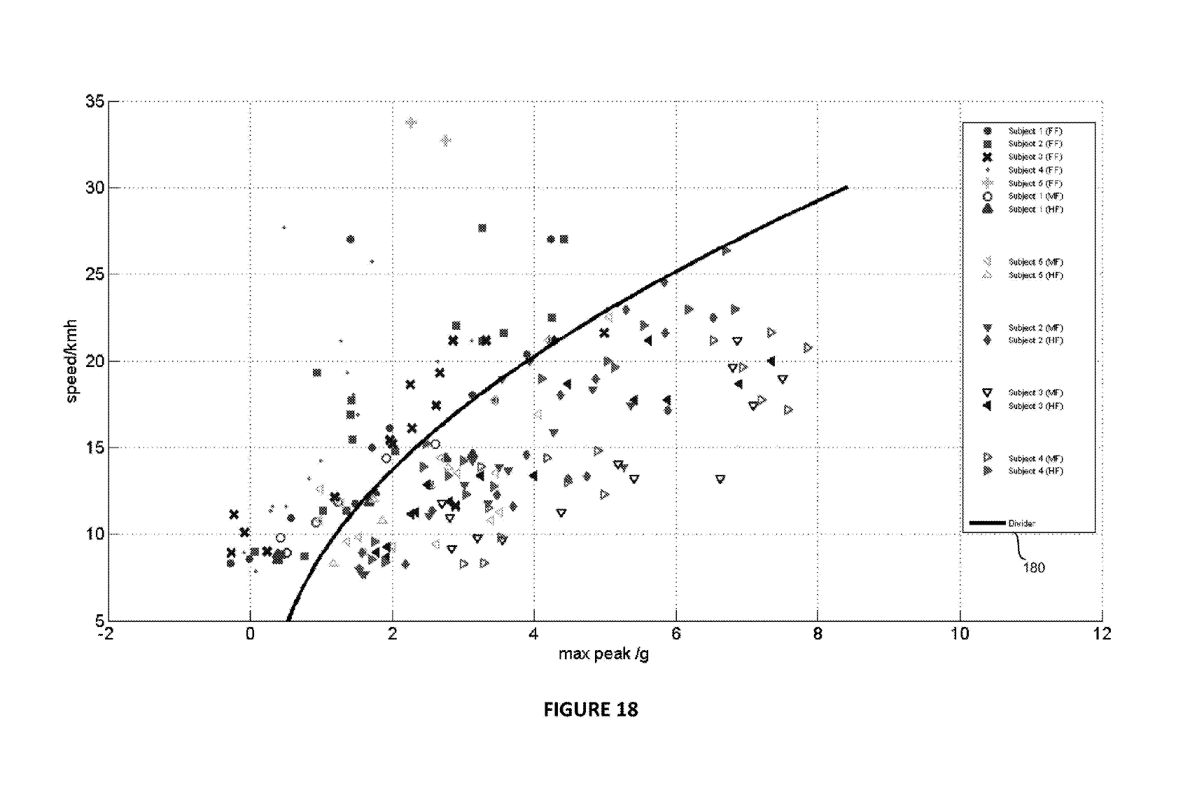

Foot strike type is relevant to maintaining good performance and injury prevention. Hind-foot runners show less loading at the ankle than fore-foot runners, however, fore-foot strikers have less loading at the knees. Hence, if a runner has a history of problems at the knee, he/she can change to a more fore-foot strike pattern. Conversely, a fore-foot runner with Achilles problems for example should move to a rear-foot striking to avoid load at the ankle. FIG. 18 shows a scatter plot between positive acceleration at Flat Foot (FF) event (refer FIG. 1b) and speeds measured by timing gates. On the left side of the non-linear divider, five subjects did fore-foot running, whereas on the right side, all subjects did mid-foot (MF) and hind-foot (HF) running. The subjects 1-5 and events (FF, MF, HF) are marked with respective symbols as shown in legend 180. For example, subject 1 (MF) is marked with a small circle. The equation for the divider is:

Acc_{Div}=0.01*speed²−0.35 (22)

wherein speed is in km/h and Acc_{Div} is in g's.

### Ground Reaction Forces

A method and apparatus for measuring ground reaction forces is disclosed in Applicants co-pending PCT application AU2013/000814 referred to herein. In the latter application it was shown that correlation components between acceleration data and reaction force are essentially non-linear when taking into account variations in speed (6 km/h-26 km/h) and in body mass of subject 12. Hence, it was shown that acceleration data may be correlated with peak ground reaction force according to the following equation:

GRF_{Peak}(acc,m)=a(m)*[log₂(acc+b)]+c(m) (23)

wherein:
“a” denotes a slope of a logarithmic function and is typically a linear function of the body mass m of subject 12;
“b” is a fixed coefficient (typically set to 1) to compensate accelerations lower than 0 g;
“c” denotes an offset associated with the logarithmic function and typically is a linear function of body mass m of subject 12;

a(m)=4.66*m−76.60; and

c(m)=24.98*m−566.8

The two coefficients a(m) and c(m) may be assumed to be substantially linear functions with respect body mass m of subject 12. Initially, for each subject 12, a linear relationship between peak ground reaction forces and the peak accelerations may be estimated. For each equation (one per subject) gain and offsets may be modelled as a function of body mass of each subject. It was found that when such modelling was performed substantially linear approximation between individual gains and offsets correlated highly with the respective body masses leading to reduced error in estimating the ground reaction force.

Finally, it is to be understood that various alterations, modifications and/or additions may be introduced into the constructions and arrangements of parts previously described without departing from the spirit or ambit of the invention.

1. An apparatus for monitoring, measuring and/or estimating metrics associated with Quality of a dynamic activity of a body or body part of a vertebral mammal, said apparatus including:
at least one inertial sensor for measuring relative to a first frame of reference acceleration and/or rotation data indicative of said Quality of a dynamic activity and for providing said acceleration and/or rotation data;
a memory device adapted for storing said acceleration and/or rotation data; and
a processor adapted for processing said acceleration and/or rotation data to evaluate one or more biomechanical metrics associated with Quality of said dynamic activity that correlates to said data.

2. The apparatus according to claim 1 including a magnetic field sensor for measuring a magnetic field around said body or body part and for providing data indicative of said magnetic field.

3. The apparatus according to claim 1 wherein said dynamic activity includes walking and/or running.

4. The apparatus Apparatus according to claim 1 wherein said processor is configured to execute at least one algorithm for evaluating said one or more biomechanical metrics associated with quality of said dynamic activity.

5. The apparatus according to claim 4 wherein said at least one algorithm is adapted to evaluate the or each biomechanical metric based on features of a signal detected by a Wavelet transform of said data.

6. The apparatus according to claim 5 wherein said Wavelet Transform is adapted to detect local features in a time-domain of a signal measured by the at least one inertial sensor.

7. The apparatus according to claim 6 wherein said local features include specific peaks, troughs and/or slope of said signal being features related to known events, such as heel strike, toe off and/or knee deviation.

8. The apparatus according to claim 5 wherein said Wavelet Transform is adapted to decompose said signal into approximation decompositions and detail decompositions associated with said local features.

9. The apparatus according to claim 8 wherein said approximation decompositions are used to locate a low frequency region of said dynamic activity.

10. The apparatus according to claim 8 wherein said detail decompositions are used to detect peaks and troughs in said signal.

11. The apparatus according to claim 1 wherein said metrics associated with quality of said dynamic activity include a measure of airborne time, speed, vertical, medio-lateral and anterior-posterior speeds, displacement, distance, stride length, stride rate, knee height, knee deviation, ground contact time, foot strike type, minimum toe clearance, acceleration and/or angular rate of change of said body or body part, vertical, horizontal, rotational 3D forces, timing of forces and impact and vibration applied to and/or experienced by said body or body part.

12. The apparatus according to claim 1 wherein said biomechanical metrics are used to provide a scoring system for quality of the dynamic activity.

13. The apparatus according to claim 12 wherein two or more biomechanical metrics are used in combination to provide a score or measure of said quality of a dynamic activity of a body or body part of a vertebral mammal.

14. The apparatus according to claim 1 wherein the or each metric associated with quality of said dynamic activity is assessed with reference to a preferred range or threshold of values.

15. Apparatus according to claim 1 wherein said at least one inertial sensor includes an accelerometer.

16. The apparatus according to claim 15 wherein said accelerometer is adapted for measuring acceleration along one or more orthogonal axes.

17. The apparatus according to claim 1 wherein said at least one inertial sensor includes a gyroscope and/or a magnetometer.

18. The apparatus according to claim 1 wherein said body of said mammal includes tibias and the at least one inertial sensor includes a wireless acceleration sensor adapted to be placed on each tibia.

19. The apparatus according to claim 1 wherein said at least one inertial sensor includes an analog to digital (A to D) converter for converting analog data to a digital domain.

20. The apparatus according to claim 19 wherein said A to D converter is configured to convert an analog output from said at least on inertial sensor to digital data prior to storing said data.

21. The apparatus according to claim 1 including means for providing feedback to a subject being monitored.

22. The apparatus according to claim 1 wherein said algorithm is adapted to transform said data from said first frame of reference to a second frame of reference in which said body part performs a movement.

23. The apparatus according to claim 1 wherein said at least on inertial sensor includes a rotation sensor.

24. The apparatus s according to claim 23 wherein said rotation sensor includes a gyroscope adapted for measuring rotation around one or more orthogonal axes.

25. The apparatus according to claim 1 wherein said algorithm is adapted to integrate rotation data over a period of time to provide an angular displacement (θ).

26. A method for monitoring, measuring and/or estimating metrics associated with Quality of a dynamic activity of a body or body part of a vertebral mammal, said method including:
using at least one inertial sensor to measure relative to a first frame of reference acceleration and/or rotation data indicative of said Quality of a dynamic activity and to provide said acceleration and/or rotation data;
storing said acceleration and/or rotation data in a memory device; and
processing said acceleration and/or rotation data by a processor to evaluate one or more biomechanical metrics associated with Quality of said dynamic activity that correlates to said data.

27. A method according to claim 26 including using a magnetic field sensor to measure a magnetic field around said body or body part and to provide data indicative of said magnetic field.

28. A method according to claim 26 wherein said dynamic activity includes walking and/or running.

29. A method according to claim 26 wherein said processor is configured to execute at least one algorithm for evaluating said one or more biomechanical metrics associated with quality of said dynamic activity.

30. A method according to claim 29 wherein said at least one algorithm is adapted to evaluate the or each biomechanical metric based on features of a signal detected by a Wavelet transform of said data.

31. A method according to claim 30 wherein said Wavelet Transform is adapted to detect local features in a time-domain of a signal measured by the at least one inertial sensor.

32. A method according to claim 31 wherein said local features include specific peaks, troughs and/or slope of said signal being features related to known events, such as heel strike, toe off and/or knee deviation.

33. A method according to claim 31 wherein said Wavelet Transform is adapted to decompose said signal into approximation decompositions and detail decompositions associated with said local features.

34. A method according to claim 33 wherein said approximation decompositions are used to locate a low frequency region of said dynamic activity.

35. A method according to claim 33 wherein said detail decompositions are used to detect peaks and troughs in said signal.

36. A method according to claim 26 wherein the or each metric associated with quality of said dynamic activity includes a measure of airborne time, speed, vertical, medio-lateral and anterior-posterior speeds, displacement, distance, stride length and/or stride rate, knee height, knee deviation, ground contact time, foot strike type, minimum toe clearance, acceleration and/or angular rate of change of said body or body part, vertical, horizontal, rotational 3D forces, timing of forces and impact and vibration applied to and/or experienced by said body or body part.

37. A method according to claim 26 wherein said biomechanical metrics are used to provide a scoring system for quality of the dynamic activity.

38. A method according to claim 37 wherein two or more biomechanical metrics are used on combination to provide a score or measure of said quality of a dynamic activity of a body or body part of a vertebral mammal.

39. A method according to claim 26 wherein the or each metric associated with quality of said dynamic activity is assessed with reference to a preferred range or threshold of values.

40. A method according to claim 26 wherein said at least one inertial sensor includes an accelerometer.

41. A method according to claim 40 wherein said accelerometer is adapted for measuring acceleration along one or more orthogonal axes.

42. A method according to claim 26 wherein said at least one inertial sensor includes a gyroscope and/or a magnetometer.

43. A method according to claim 26 wherein said body of said mammal includes tibias and the at least one inertial sensor includes a wireless accelerometer adapted to be placed on each tibia.

44. A method according to claim 26 wherein said at least one inertial sensor includes an analog to digital (A to D) converter for converting analog data to a digital domain.

45. A method according to claim 44 wherein said A to D converter is configured to convert an analog output from said at least one inertial sensor to digital data prior to storing said data.

46. A method according to claim 26 including means for providing feedback of said deviation to a subject being monitored.

47. A method according to claim 26 wherein said algorithm is adapted to transform said data from said first frame of reference to a second frame of reference in which said body part performs a movement.

48. A method according to claim 26 wherein said at least one inertial sensor includes a rotation sensor.

49. A method according to claim 48 wherein said rotation sensor includes a gyroscope adapted for measuring rotation around one or more orthogonal axes.

50. A method according to claim 26 wherein said algorithm is adapted to integrate said rotation data over a period of time to provide an angular displacement (θ).
